(19) United States
(12) Patent Application Publication
(10) Pub. No.: US 2013/0253516 A1
(43) Pub. Date: Sep. 26, 2013

(54) OCCIPITAL PLATE

(71) Applicant: John L Mackall, Arlington, VA (US)

(72) Inventor: John L Mackall, Arlington, VA (US)

(21) Appl. No.: 13/429,203

(22) Filed: Mar. 23, 2012

(51) Int. Cl. A61B 17/80

(52) U.S. Cl. 606/70

(57) ABSTRACT

An occipital plate for use in an occipitocervical fixation procedure to stabilize the base of a patient's skull with respect to the patient's neck. The occipital plate of the invention is made up of a middle portion having left and right sides, and left and right hinged legs extending outward in opposite directions from the left and right sides of the middle portion. Each of the left and right hinged legs uses a hinge mechanism to secure a spinal rod to the occipital plate.

### CROSS-REFERENCE TO RELATED APPLICATIONS

Not Applicable.

### STATEMENT REGARDING FEDERALLY SPONSORED RESEARCH OR DEVELOPMENT

Not Applicable.

### FIELD OF THE INVENTION

This invention relates to medical devices. More specifically, the invention is directed to an occipital plate.

### BACKGROUND OF THE INVENTION

As noted in U.S. Pat. No. 7,695,500 issued to Markworth, there are many occipital plate implants on the market today. Some implants have through-holes and must be preloaded on the rod. Others have top loading sockets similar to polyaxial screws which allow bent rods to be anchored to the plate. The most simple, but generally most difficult, to use form is that of a rod that smoothly tapers to an occipital plate that must be bent and contoured to match highly varied anatomy. All of these iterations have at most one to two degrees of freedom and typically require long preparation time to ensure a proper bend. This makes the implants both difficult to connect to the longitudinal rod member and the occipital plateau without putting stress on the atlantoaxial joint.

Accordingly, there remains a need for an improved occipital plate.

### SUMMARY OF THE INVENTION

An occipital plate for use in an occipitocervical fixation procedure to stabilize the base of a patient's skull with respect to the patient's neck. The occipital plate of the invention is made up of a middle portion having left and right sides, and left and right hinged legs extending outward in opposite directions from the left and right sides of the middle portion. Each of the left and right hinged legs uses a hinge mechanism to secure a spinal rod to the occipital plate.

### BRIEF DESCRIPTION OF THE DRAWINGS

FIG. 1 is an environmental perspective view of an occipital plate, according to the present invention.

FIG. 2 shows a front view of the occipital plate of FIG. 1 but with left and right hinged legs in an open configuration.

FIG. 3 shows a front view of the occipital plate of FIG. 1 but with left hinged leg in a closed configuration and a right hinged leg in a partially closed configuration.

FIG. 4 shows a front view of the occipital plate of FIG. 1 but with left and right hinged legs in a closed configuration.

FIG. 5 shows a rear view of the occipital plate of FIG. 4.

FIG. 6 shows a top side view of the occipital plate of FIG. 4.

FIG. 7 shows a bottom side view of the occipital plate of FIG. 4.

FIG. 8 shows a front view of an occipital plate with first and second hinged legs comprising high friction front surfaces, according to the invention.

FIG. 9 shows an exploded view of the occipital plate of FIG. 1, according to the invention.

FIGS. 10 through 15 show various views of an occipital plate according to the invention.

FIG. 16 shows a table (Table 1) listing part numbers.

The remaining Figures show further views of an occipital plate according to the invention.

Similar reference characters denote corresponding features consistently throughout the attached drawings.

### DETAILED DESCRIPTION OF THE PREFERRED EMBODIMENTS

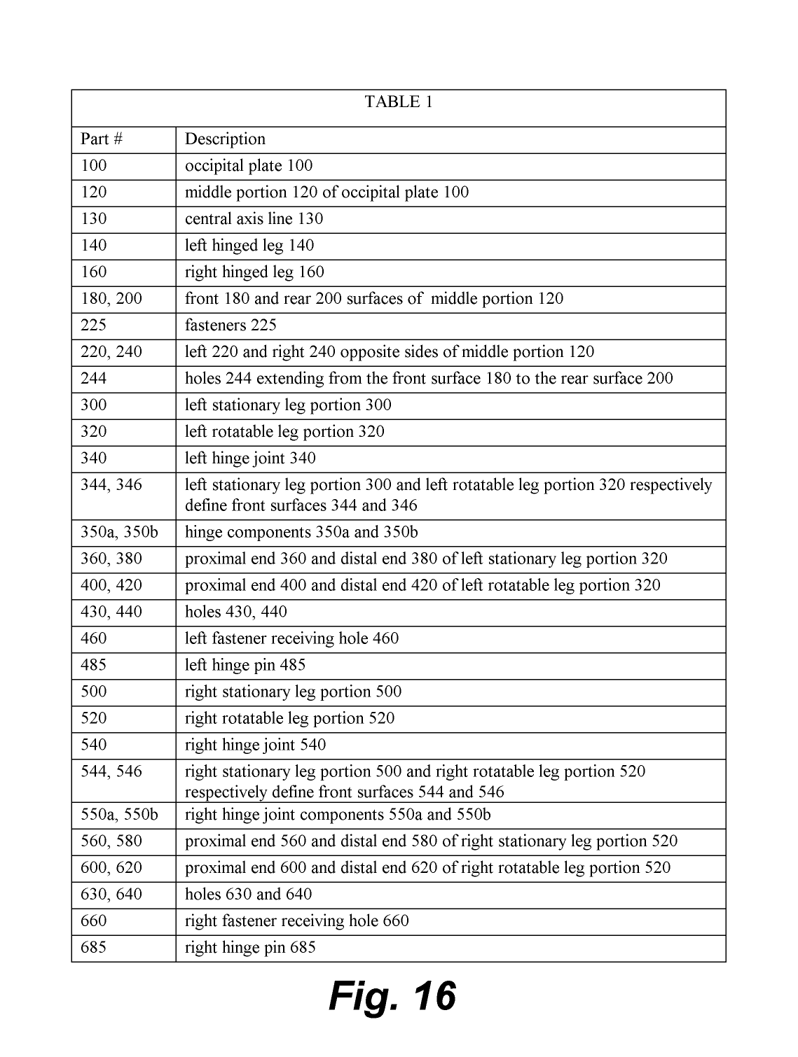

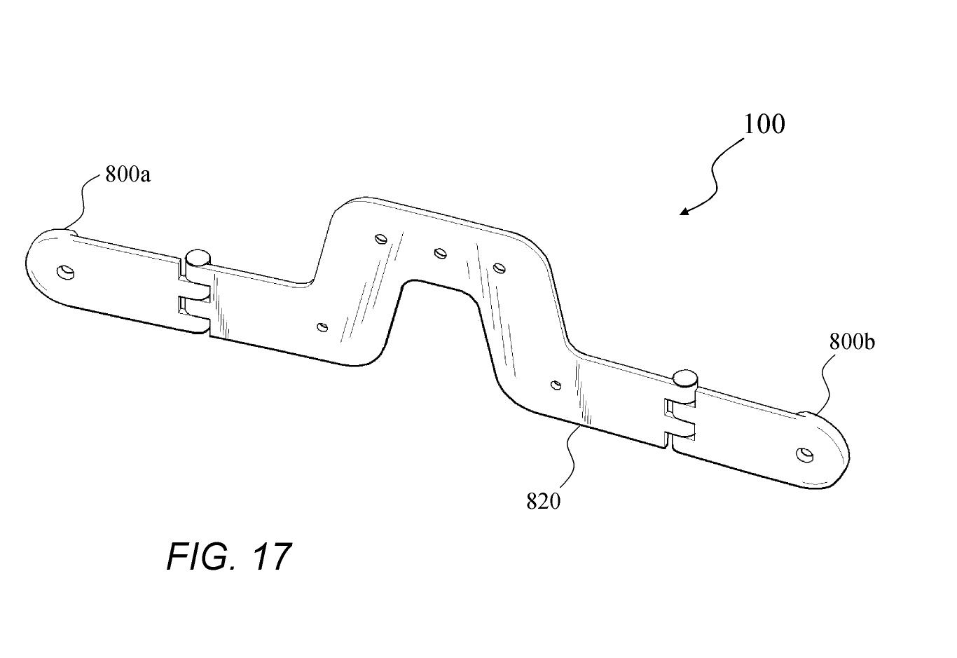

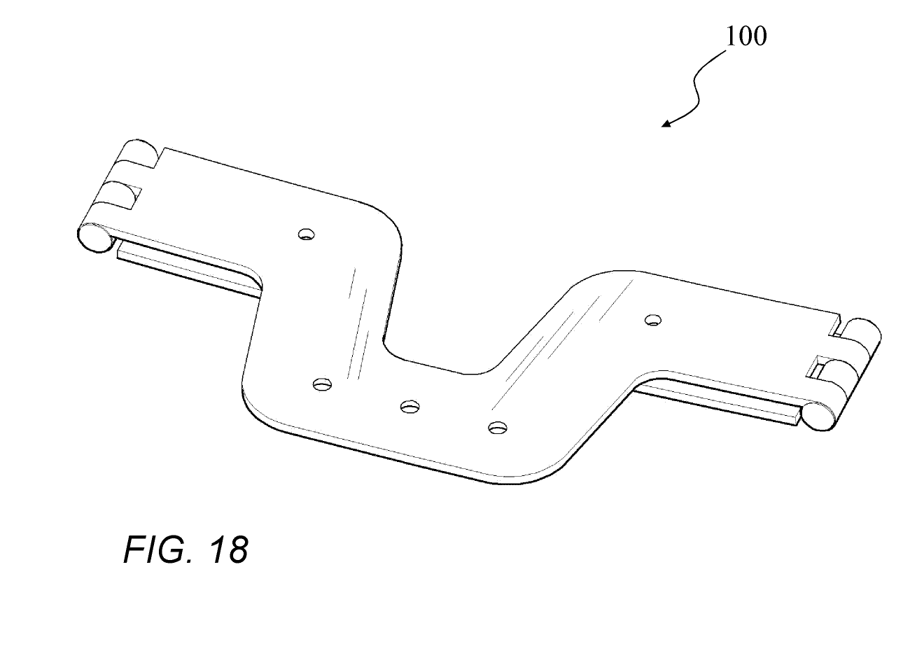

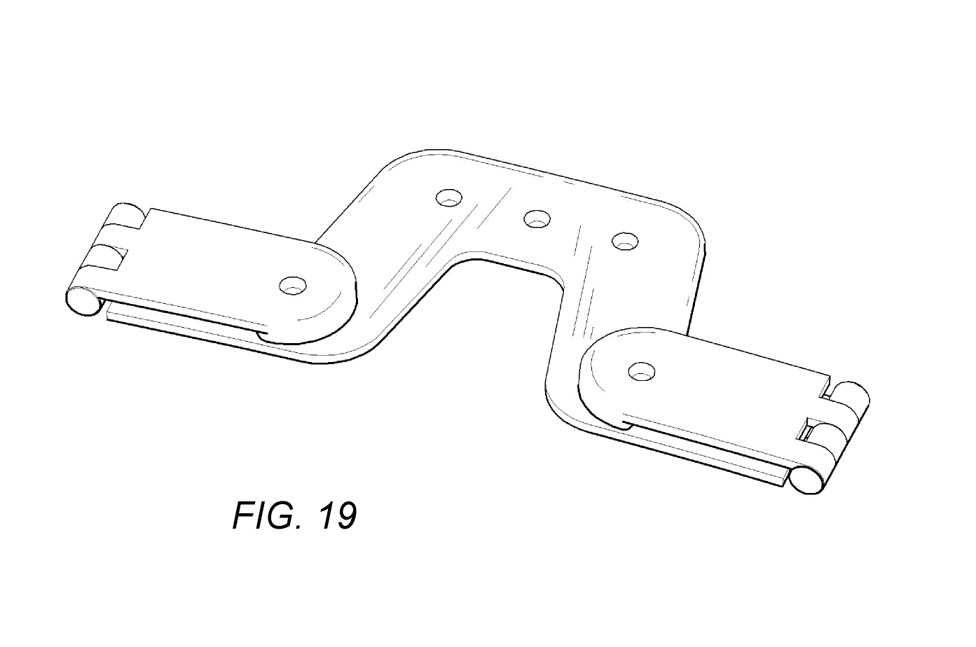

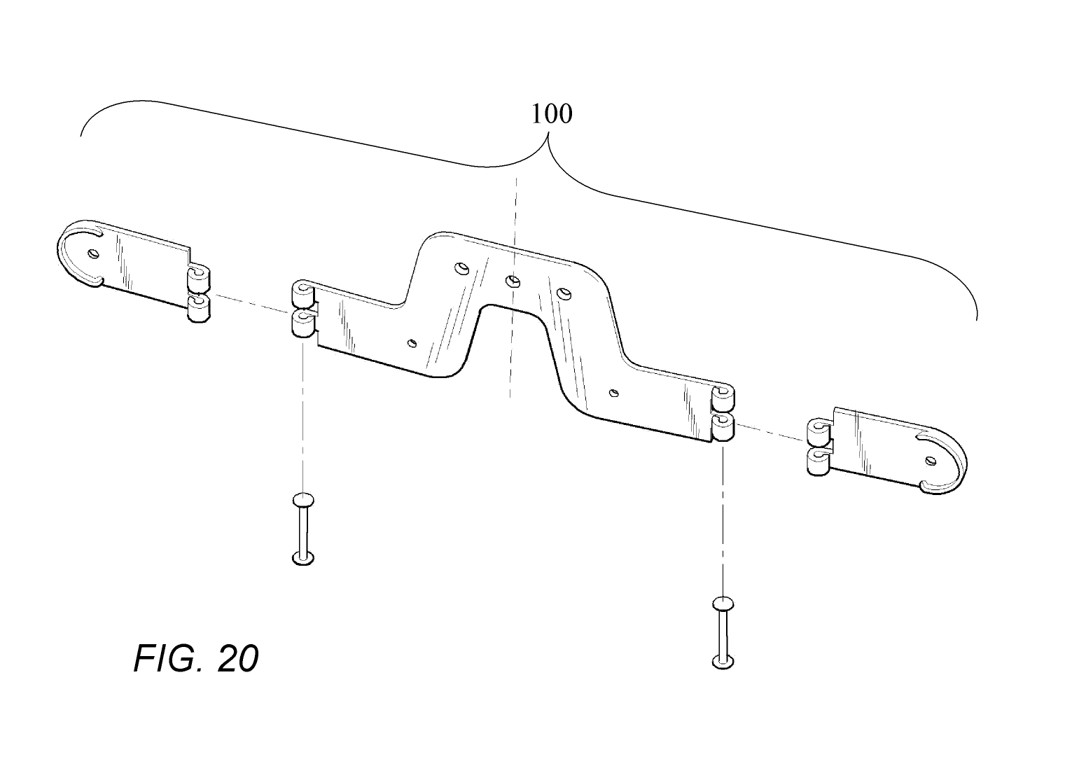

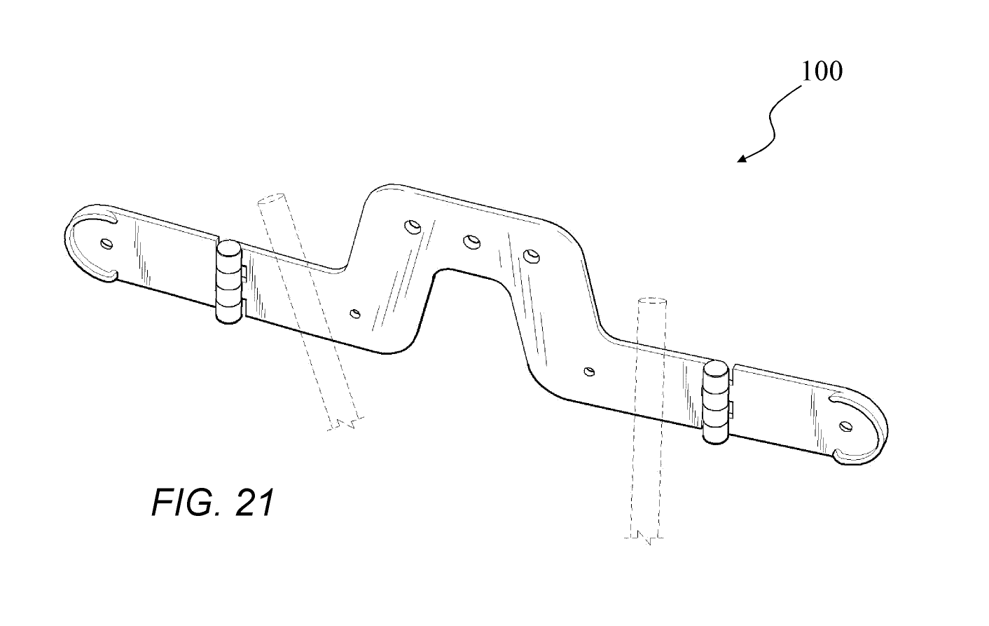

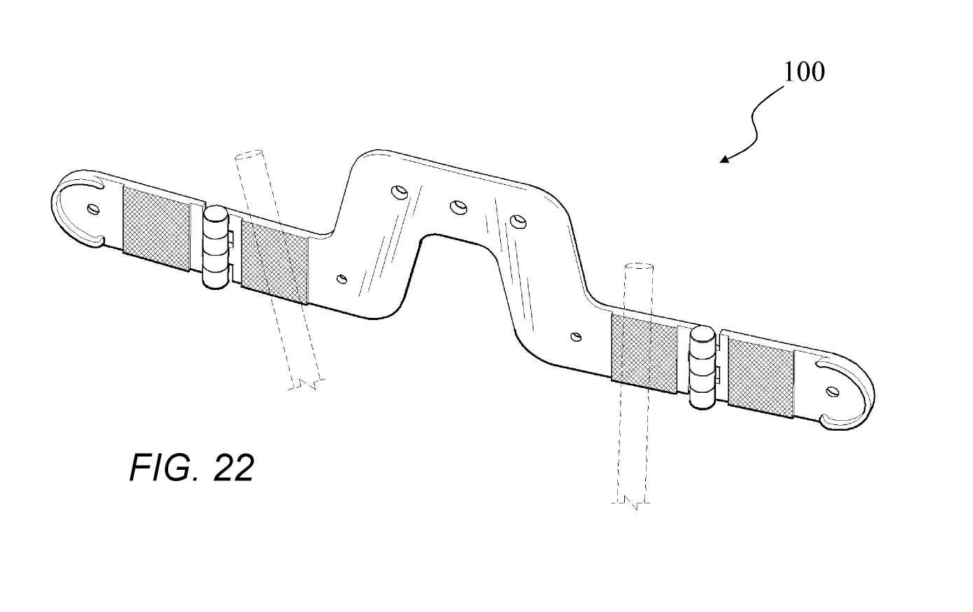

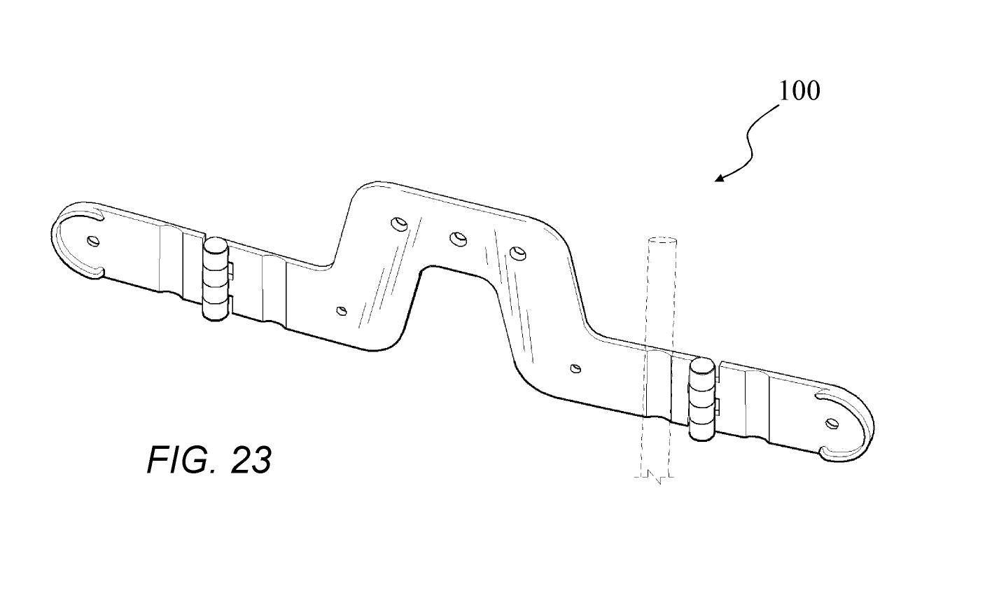

This invention relates to medical devices. More specifically, the invention is directed to an occipital plate 100 for use in an occipitocervical fixation procedure. The occipital plate 100 and its parts can be made out of any suitable material such as, but not limited to, titanium, tungsten, and stainless steel, alone or in combination. Part numbers are listed in Table 1 (shown in FIG. 16).

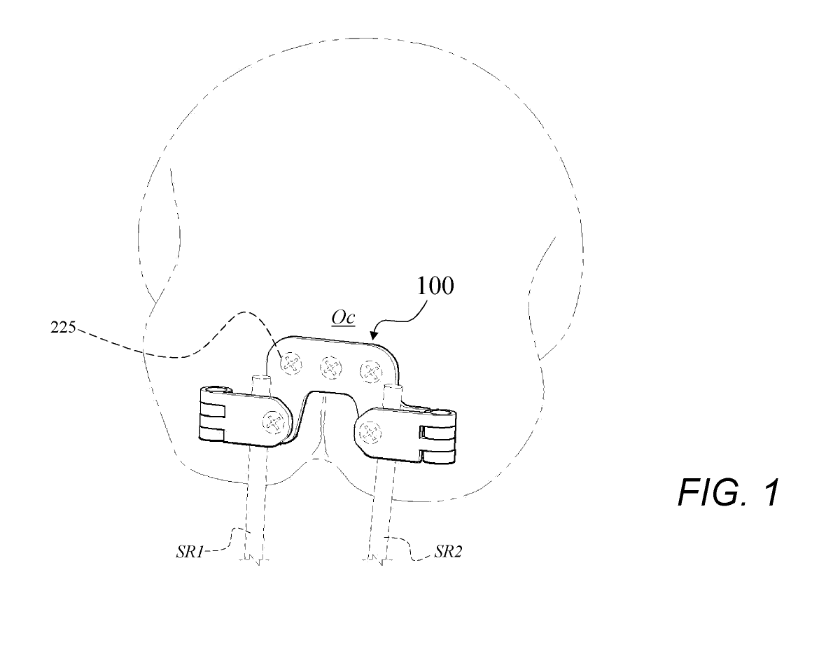

The occipital plate 100 comprises a middle portion 120, a left hinged leg 140, and a right hinged leg 160. The occipital plate 100 defines a central axis line 130. While not necessary it is preferred that the occipital plate 100 exhibits 2-fold symmetry about central axis line 130. The middle portion 120 defines front 180 and rear 200 surfaces, and left 220 and right 240 opposite sides. Holes 244 extend from the front surface 180 through to the rear surface 200 of the middle portion 120 are provided for receiving bone fasteners 225 for fixation of occipital plate 100 to the occiput Oc, i.e., the posterior (back) portion of a patient's head (see FIG. 1). The holes 244 can be in the form of countersunk screw holes. The occipital plate 100 can be curved for facilitating attachment to the lower posterior curve of the skull known as the occipital region (labeled as “Oc” in FIG. 1).

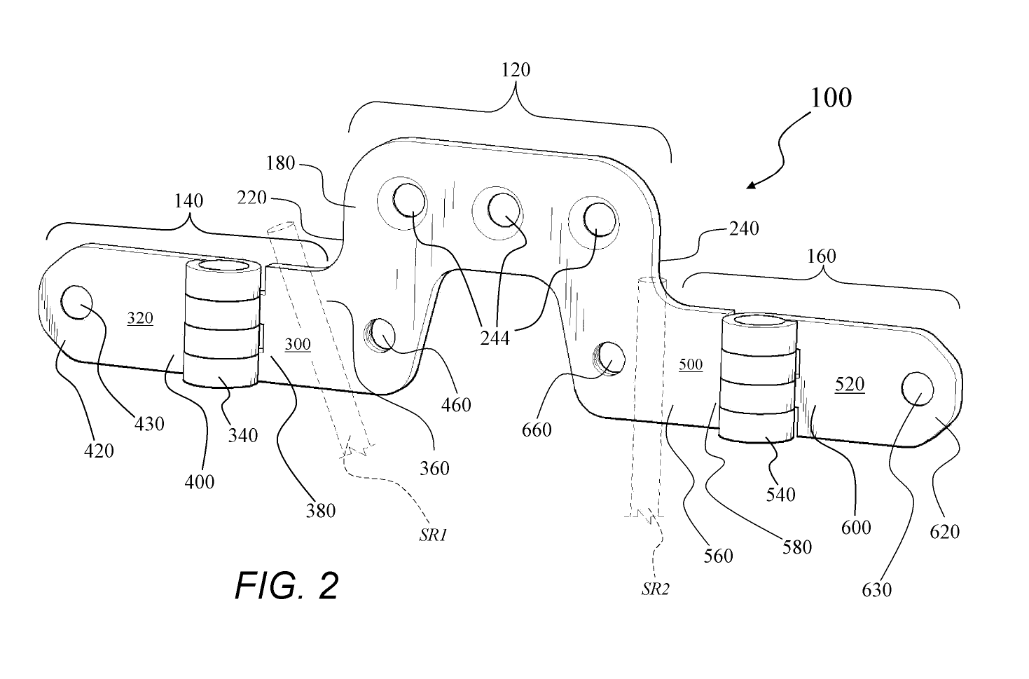

Left and right hinged legs 140 and 160 respectively extend outward from left 220 and right 240 opposite sides of the middle portion 120. The left hinged leg 140 comprises a left stationary leg portion 300, a left rotatable leg portion 320, and a left hinge joint 340. The left stationary leg portion 300 defines proximal end 360 and distal end 380 thereof. Left rotatable leg portion 320 defines proximal end 400 and distal end 420 thereof. The distal end 420 defines through-hole 430, which can be a countersunk screw hole. The terms “proximal” and “distal” are used with respect to the middle portion 120 when the hinged legs of occipital plate 100 are in an open configuration as shown in FIG. 2. The left hinge joint 340 can take any suitable form. In one embodiment the left hinge joint 340 comprises tubular hinge components 350a and 350b and left hinge pin 485.

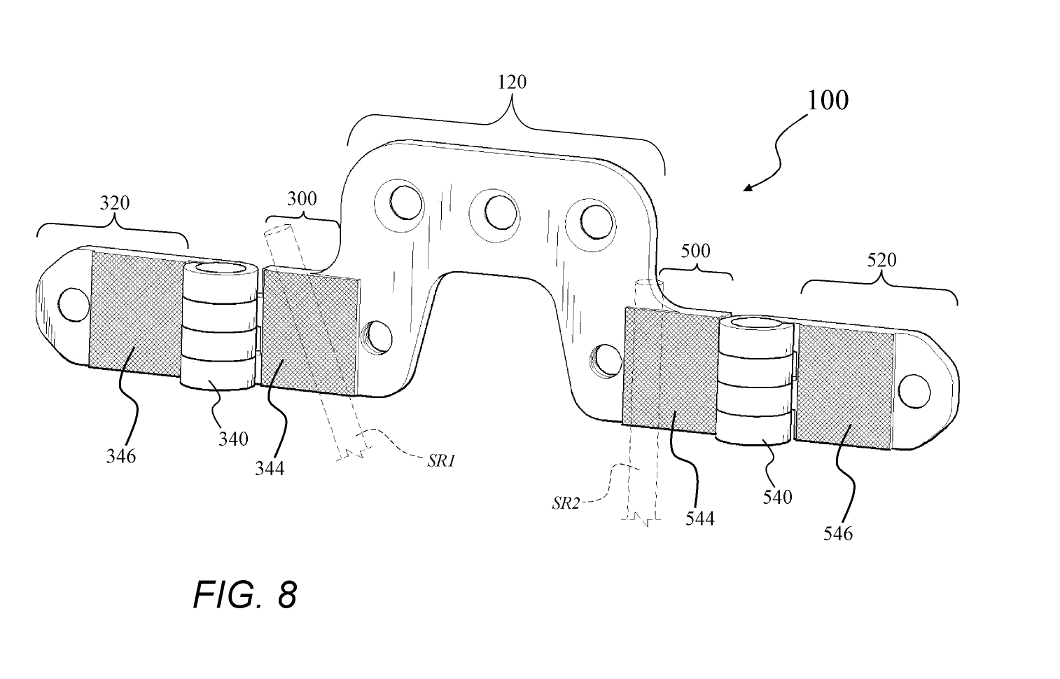

The left stationary leg portion 300 and left rotatable leg portion 320 are connected via the left hinge joint 340. More specifically, the distal end 380 of left stationary leg portion 300 and the proximal end 400 of left rotatable leg portion 320 are each connected to hinge joint 340. As shown, for example, in FIG. 2 the proximal end 360 of the left stationary leg portion 300 is integral with the left opposite side 220 of the middle portion 120 whereas the distal end 380 of the left stationary leg is operatively connected to the left hinge joint 340. The left rotatable leg portion 320 pivots about left hinge joint 340. The amount of rotation is sufficient to allow the securement of a spinal rod (represented by first spinal rod shown as “SR1” in FIG. 8) to the left hinged leg 140.

The term “front surface” refers to the surface which during and post surgery is opposed to the one (the rear surface) that faces the bone of a patient's occipital region. The term “rear surface” refers to the surface which during and post surgery faces the bone of a patient's occipital region. The left stationary leg portion 300 and left rotatable leg portion 320 respectively define front surfaces 344 and 346. The front surfaces 344 and 346 can be machined to provide high friction surfaces as shown in FIG. 8; such surfaces are preferably knurled, in order to bite into the surface of a spinal rod.

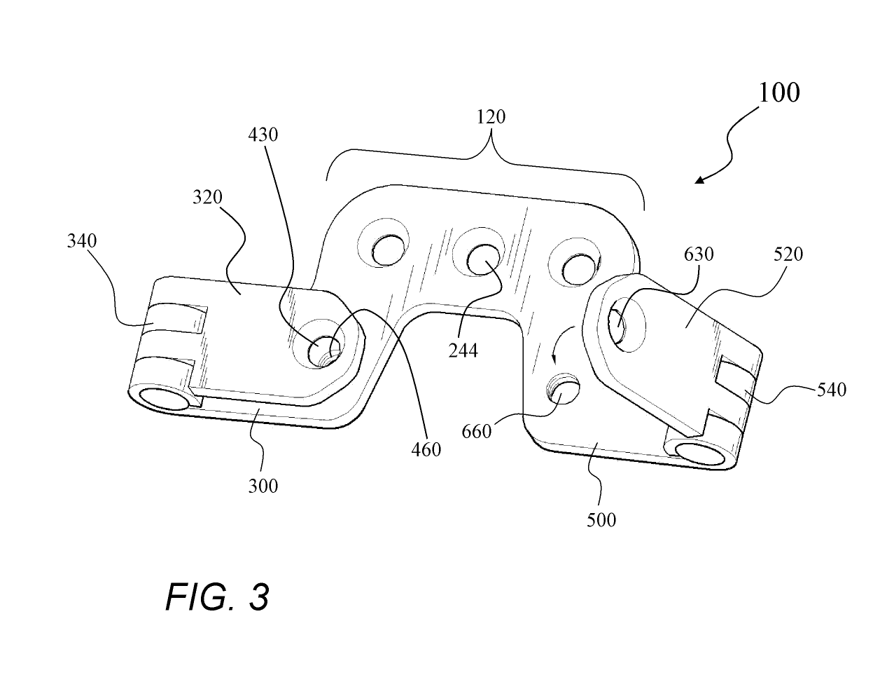

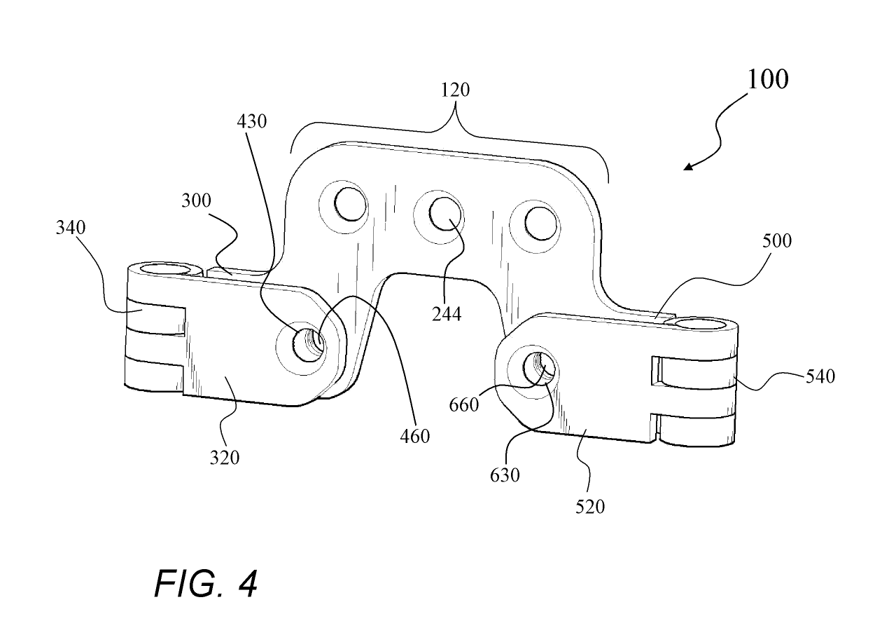

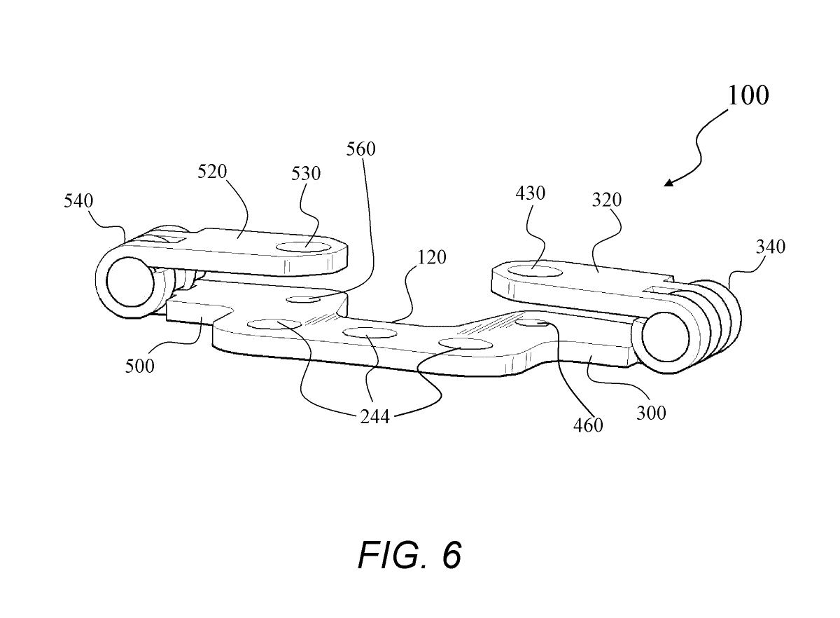

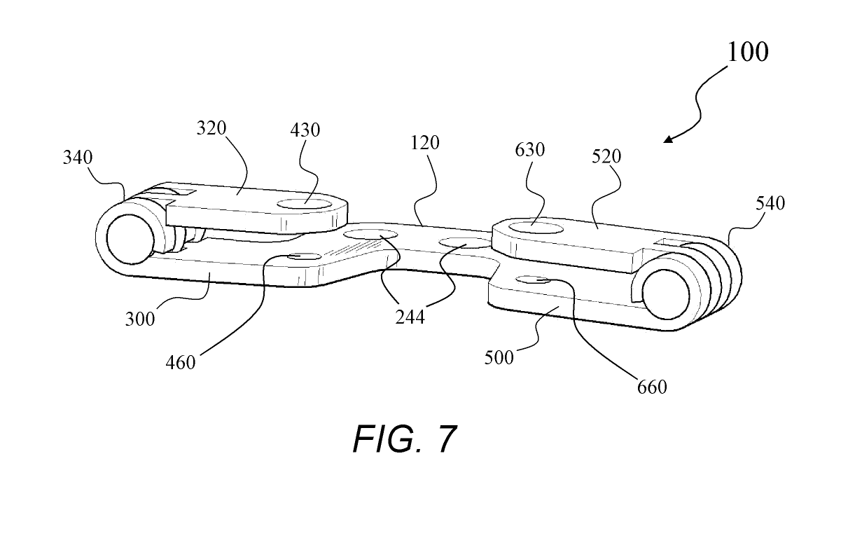

In one embodiment the left rotatable leg portion 320 of the left hinged leg 140 is of sufficient length to allow its distal end 420 to be directly fastened to the middle portion 120 of occipital plate 100 (see, e.g., FIGS. 2 through 7). In this embodiment the middle portion 120 defines a complementary left fastener receiving hole 460. During surgery the left rotatable leg portion 320 is rotated about the hinge joint 340 until the through hole 430 of distal end 420 is aligned over the complementary left fastener receiving hole 460 whereupon a fastener, such as a locking screw, is affixed into hole 460 via through hole 430 to secure the left rotatable leg portion 320 to the middle portion 120. Fasteners are shown in FIG. 1.

The purpose of securing the distal end 420 of the left hinged leg 140 to the middle portion 120 is to clamp a spinal rod (represented by alpha-numeric label SR1 in, e.g. FIGS. 1 and 2) to the occipital plate 100. This procedure provides flexibility to the surgeon who is not limited to the specific location of a prior art seat as typically found on a prior art occipital plates. Moreover, rods can be secured at various angles to the left hinged leg 140 as shown in FIG. 2.

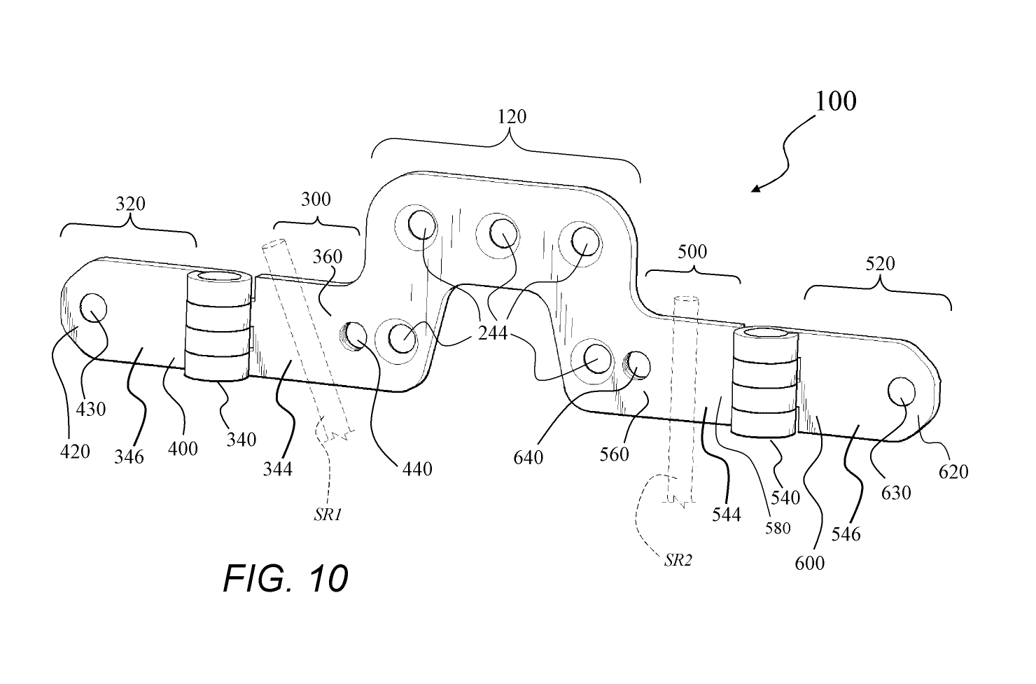

In another embodiment, the proximal end 360 of left stationary leg portion 300 defines a left fastener receiving hole 440 (see FIG. 10). The fastener receiving hole 440 may be a threaded blind hole. In this embodiment a fastener, such as a locking screw, is used to fasten the distal end 420 of the left rotatable leg portion 320 to the proximal end 360 of stationary leg portion 300.

The right hinged leg 160 comprises a right stationary leg portion 500, a right rotatable leg portion 520, and a right hinge joint 540. The right stationary leg portion 500 defines proximal end 560 and distal end 580 thereof. Right rotatable leg portion 520 defines proximal end 600 and distal end 620 thereof. The proximal end 600 defines through-hole 630, which can be a countersunk screw hole. The terms “proximal” and “distal” are used with respect to the middle portion 120 when the hinged legs of occipital plate 100 are in an open configuration as shown in FIG. 2. The right hinge joint 540 can take any suitable form. In one embodiment the right hinge joint 540 comprises tubular hinge components 550a and 550b and right hinge pin 685.

The right stationary leg portion 500 and right rotatable leg portion 520 are connected via the right hinge joint 540. More specifically, the distal end 580 of right stationary leg portion 500 and the proximal end 600 of right rotatable leg portion 520 are each connected to hinge joint 540. As shown, for example, in FIG. 2 the proximal end 560 of the right stationary leg portion 500 is integral with the right opposite side 220 of the middle portion 120 whereas the distal end 580 of the right stationary leg is operatively connected to the right hinge joint 540. The right rotatable leg portion 520 pivots about right hinge joint 540. The amount of rotation is sufficient to allow the securement of a spinal rod (represented by second spinal rod shown as “SR2” in FIG. 8) to the right hinged leg 160.

In one embodiment the right rotatable leg portion 520 of the right hinged leg 160 is of sufficient length to allow its distal end 620 to be directly fastened to the middle portion 120 of occipital plate 100 (see, e.g., FIGS. 2 through 7). In this embodiment the middle portion 120 defines a complementary right fastener receiving hole 660. During surgery the right rotatable leg portion 520 is rotated about the hinge joint 540 until the through hole 630 of distal end 620 is aligned over the complementary fastener receiving hole 660 whereupon a fastener, such as a locking screw, is affixed into hole 660 via through hole 630 to secure the right rotatable leg portion 520 to the middle portion 120.

The purpose of securing the distal end 620 of the right hinged leg 160 to the middle portion 120 is to clamp a spinal rod (represented by alpha-numeric label SR2 in, e.g., FIGS. 1 and 2) to the occipital plate 100. This procedure provides flexibility to the surgeon who is not limited to the specific location of a prior art seat as typically found on a prior art occipital plates. Moreover, rods can be secured at various angles to the right hinged leg 160.

In one embodiment, the proximal end 560 of right stationary leg portion 500 defines a right fastener receiving hole 640 (see FIG. 10). The fastener receiving hole 640 may be a threaded blind hole. In this embodiment a fastener, such as a locking screw, is used to fasten the distal end 620 of the right rotatable leg portion 320 to the proximal end 560 of stationary leg portion 500.

The right stationary leg portion 500 and right rotatable leg portion 520 respectively define front surfaces 544 and 546. The front surfaces 544 and 546 can be machined to provide high friction surfaces as shown in FIG. 8; such surfaces are preferably knurled in order to bite into the surface of a spinal rod (represented by alpha-numeric label “SR2” in FIG. 8).

Referring now to the Figures with regard to which the meaning of labels and numbers shown in the Figures are summarized in Table 1 (see FIG. 16).

FIG. 1 is an environmental perspective view of an occipital plate, according to the present invention. The occipital plate 100 of the invention is shown attached by fasteners 225 to the lower posterior curve of the skull known as the occipital region Oc.

FIGS. 2, 3 and 4 respectively show a front view of the occipital plate 100 of FIG. 1 but with left and right hinged legs 140 and 160 deployed in an open, a partially closed, and a closed configuration. In this embodiment the left and right fastener receiving holes 460 and 660 are located in the middle portion 120 of occipital plate 100.

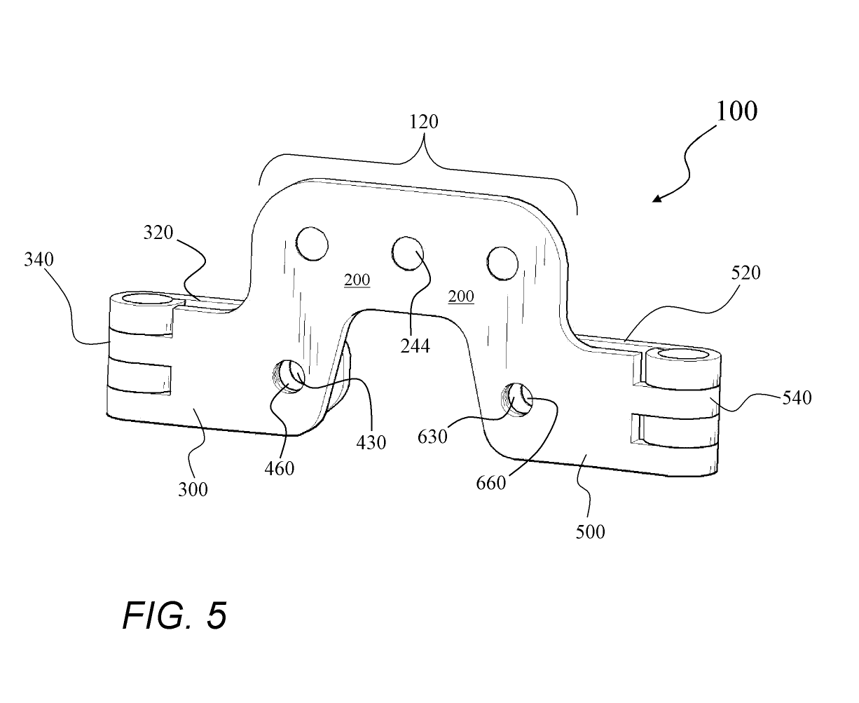

FIGS. 5, 6, and 7 respectively show rear, top side and bottom side views of the occipital plate 100 shown in closed configuration. The holes 430 and 460 are shown aligned, and likewise holes 630 and 660 are aligned and each pair of holes are able to receive a fastener to secure spinal rods SR1 and SR2 (not shown) to the right and left hinged legs 140 and 160, respectively.

FIG. 8 shows a front view of an occipital plate with first and second hinged legs 140 and 160 with high friction front surfaces. Specifically, surfaces 344, 346, 544 and 546 are machined to offer high friction surfaces to secure rods SR1 and SR2 (not shown).

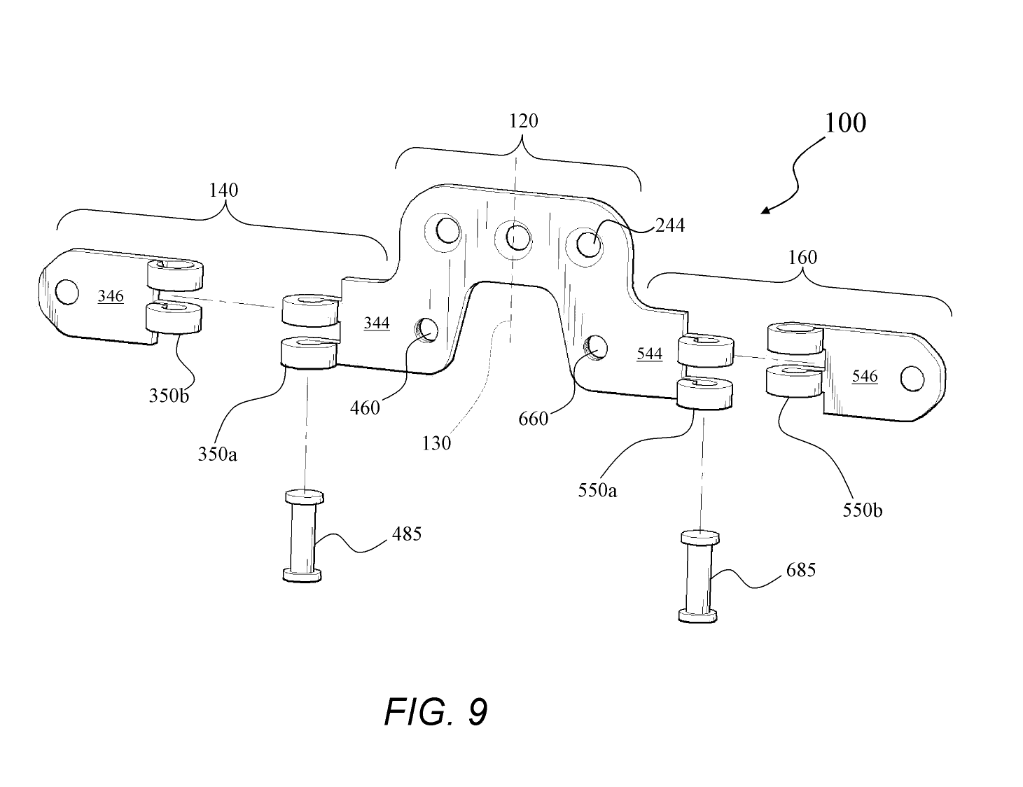

FIG. 9 shows an exploded view of the occipital plate 100 of FIG. 1, according to the invention. Left and right hinge pins 485 and 685 are shown. It should be understood that any suitable hinge mechanism can be used and is not limited to that shown in FIG. 9.

FIG. 10 shows a occipital plate 100 according to the invention wherein the proximal end 360 of left stationary leg portion 300 defines a left fastener receiving hole 440, and the proximal end 560 of right stationary leg portion 500 defines a right fastener receiving hole 640. The fastener receiving holes 440 and 640 can be threaded blind holes. In this embodiment a fastener, such as a locking screw (not shown), is used to fasten the distal end 420 of the left rotatable leg portion 320 to the proximal end 360 of stationary leg portion 300; and a fastener, such as a locking screw (not shown), is used to fasten the distal end 620 of the right rotatable leg portion 320 to the proximal end 560 of stationary leg portion 500.

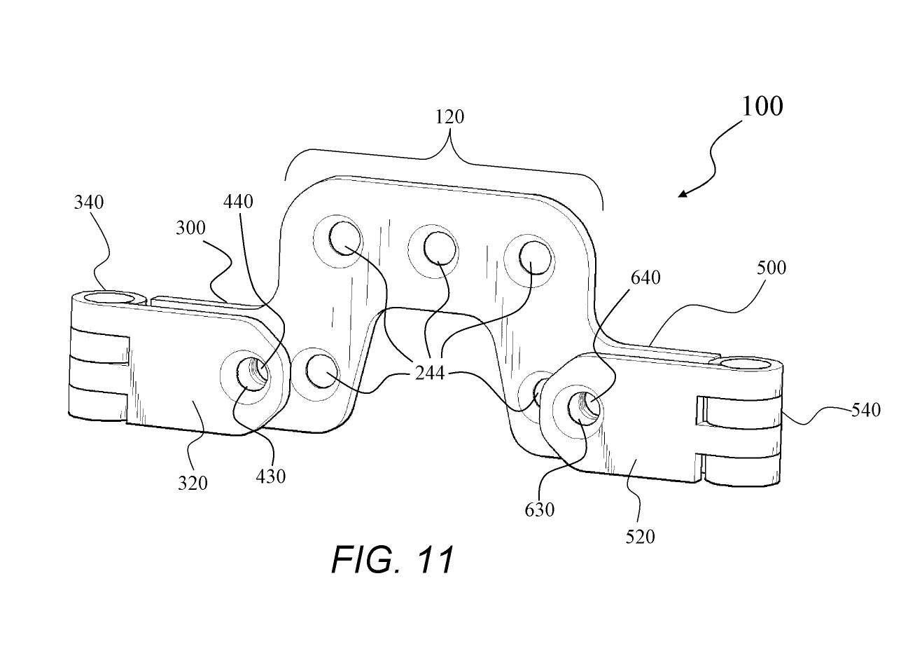

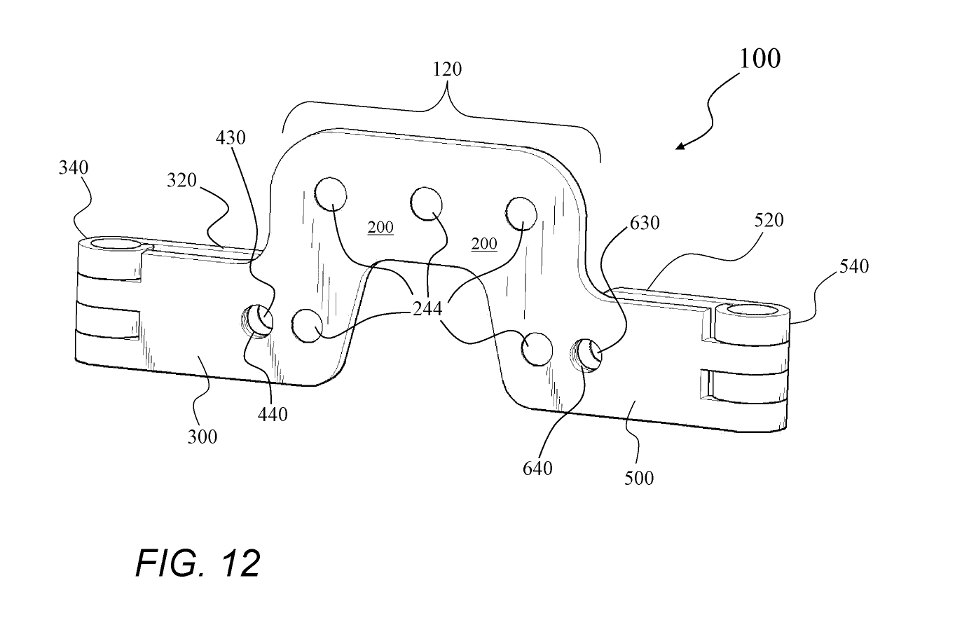

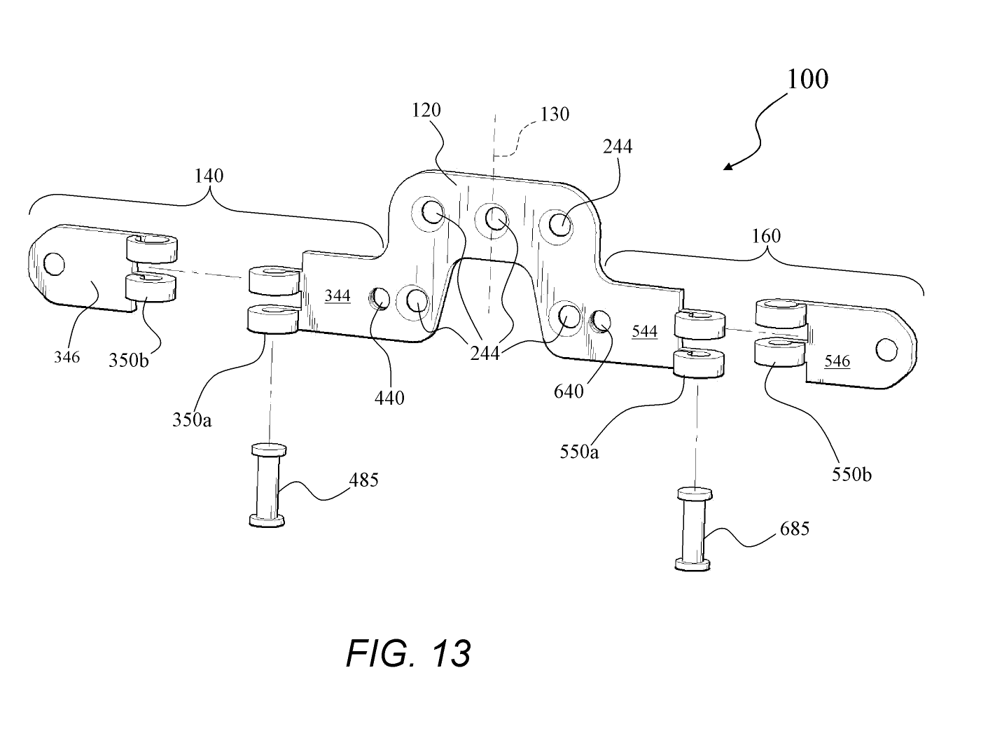

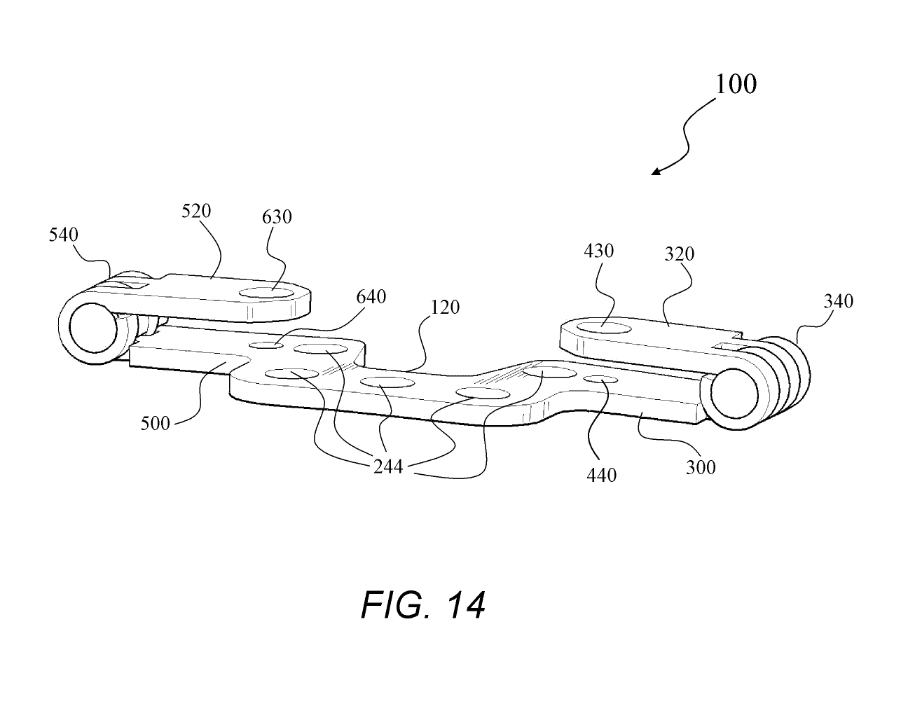

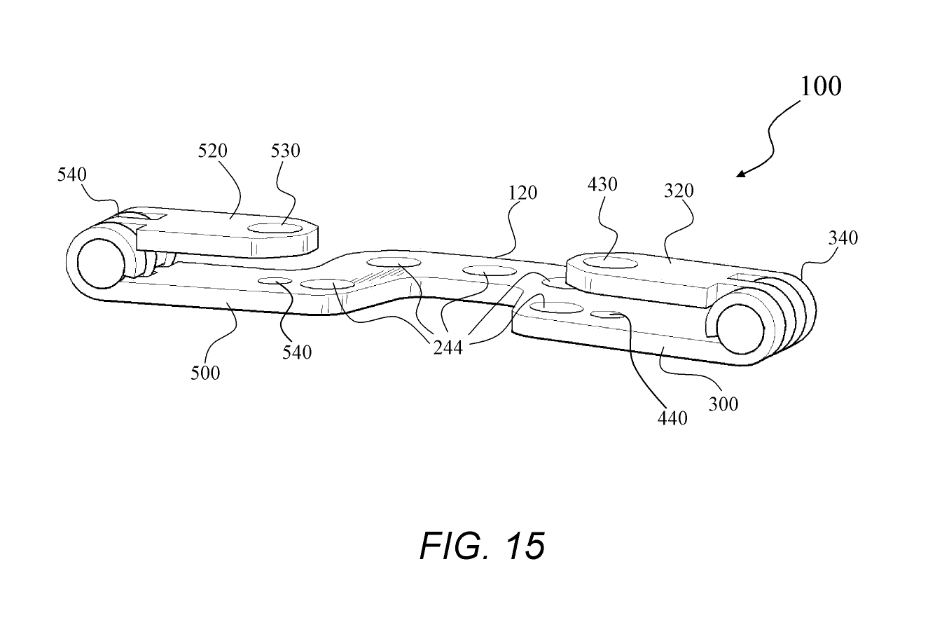

FIGS. 11 through 15 show various views of the occipital plate 100 shown in FIG. 10 according to the invention. FIG. 16 shows a table (Table 1), which comprises a list of part numbers.

In one embodiment the occipital plate 100 includes at least one curved perimeter portion 800 (e.g. see FIG. 17 where the at least one curved perimeter portion 800 is represented by alpha-numeric labels 800a and 800b); more specifically, the occipital plate 100 defines a perimeter 820, which further defines said at least one curved perimeter portion 800. The at least one curved perimeter portion 800 serves to help smooth selected edges of the occipital plate 100. During actual use of this embodiment of the occipital plate 100 the at least one curved perimeter portion 800 helps avoid snagging of a patient's tissue, such as muscle tissue, on the occipital plate 100.

The invention being thus described, it will be evident that the same may be varies in many ways by a routineer in the applicable arts. Such variations are not to be regarded as a departure from the spirit and scope of the invention.

What is claimed:

1. An occipital plate comprising:
a middle portion having opposite left and right sides, wherein the middle portion defines a plurality of through holes;
a left hinged leg for receiving and securing a first spinal rod, wherein the left hinged leg extends from the left side of the middle portion; and
a right hinged leg for receiving and securing a second spinal rod, wherein the right hinged leg extends from the right side of the middle portion.

2. An occipital plate comprising:
a middle portion having opposite left and right sides;
a left hinged leg extending from the left side of the middle portion, the left hinged leg comprising a left rotatable leg portion and a left stationary leg portion, the left rotatable leg portion and the stationary leg portion each having proximal and distal ends, wherein the proximal end of the left stationary leg portion is connected to the left side of the middle portion, and the distal end of the left stationary leg portion is connected via a left hinge to the proximal end of the left rotatable leg portion; and
a right hinged leg extending from the right side of the middle portion, the right hinged leg comprising a right rotatable leg portion and a right stationary leg portion, the right rotatable leg portion and the stationary leg portion each having proximal and distal ends, wherein the proximal end of the right stationary leg portion is connected to the right side of the middle portion, and the distal end of the right stationary leg portion is connected via a right hinge to the proximal end of the right rotatable leg portion,
wherein the distal ends of the left and right rotatable leg portions each define a through hole, and the proximal ends of the left and right stationary leg portions each define a hole therein,
wherein during normal operation of the occipital plate the distal ends of the left and right rotatable leg portions can rotate and align respectively with the proximal ends of the left and right stationary leg portions such that the holes of in the distal ends of the left and right rotatable leg portions align respectively with the holes in the proximal ends of the left and right stationary leg portions, and
wherein the middle portion defines a plurality of through holes.

3. An occipital plate comprising:
a middle portion having opposite left and right sides;
a left hinged leg extending from the left side of the middle portion, the left hinged leg comprising a left rotatable leg portion and a left stationary leg portion, the left rotatable leg portion and the stationary leg portion each having proximal and distal ends, wherein the proximal end of the left stationary leg portion is connected to the left side of the middle portion, and the distal end of the left stationary leg portion is connected via a left hinge to the proximal end of the left rotatable leg portion; and
a right hinged leg extending from the right side of the middle portion, the right hinged leg comprising a right rotatable leg portion and a right stationary leg portion, the right rotatable leg portion and the stationary leg portion each having proximal and distal ends, wherein the proximal end of the right stationary leg portion is connected to the right side of the middle portion, and the distal end of the right stationary leg portion is connected via a right hinge to the proximal end of the right rotatable leg portion,
wherein the distal ends of the left and right rotatable leg portions each define a through hole, and the proximal ends of the left and right stationary leg portions each define a hole therein and the middle portion defines a plurality of through holes including a through hole located proximate to the left side of the middle portion and a through hole located proximate to the right side of the middle portion,
wherein during normal operation of the occipital plate the distal end of the left rotatable leg portion is rotatable such that the through hole located at the distal and of the left rotatable leg portion aligns with the hole proximate to the left side of the middle portion, and
wherein during normal operation of the occipital plate the distal end of the right rotatable leg portion is rotatable such that the through hole located at the distal and of the right rotatable leg portion aligns with the hole proximate to the right side of the middle portion.

5. The occipital plate according to claim 4, wherein the occipital plate defines a perimeter, said perimeter defines at least one curved perimeter portion 800.
